(19) United States
(12) Patent Application Publication
(10) Pub. No.: US 2003/0102239 A1
(43) Pub. Date: Jun. 5, 2003

(54) Packaging and dispensing system for pouched products

(75) Inventor: Carol A. Beard, Appleton, WI (US)

Correspondence Address: Stephen E. Bondura, Dority & Manning, Attorneys at Law, P.A., P.O. Box 1449, Greenville, NC, 29602, US

(73) Assignee: KIMBERLY-CLARK WORLDWIDE, INC.

(21) Appl. No.: 09/998,376

(22) Filed: Nov. 30, 2001

(51) Int. Cl. A61L 15/00; A61B 17/06

(52) U.S. Cl. 206/440

(57) ABSTRACT

A packaging system for personal care products includes an elongated flexible strand of individual sealed pouches connected together in end-to-end fashion. Each pouch carries at least one type of personal care product therein, such as a feminine hygiene product. The strand of pouches is folded such that the individual pouches are in a stacked configuration. A separation line is defined between the individual pouches. A device is provided for maintaining the pouches in the stacked configuration during transit and storage while permitting a consumer to readily remove and separate a leading pouch from the stack. The maintaining device accommodates reduction and size of the stack as individual pouches are removed from the stack.

### BACKGROUND OF THE INVENTION

The present invention relates to the packaging and dispensing of any manner of individually pouched products, such as personal care products, and in particular to a packaging system that offers a greater degree of discretion, convenience, and portability to consumers. “Personal care products” is a relatively broad term in the art for products of a personal hygiene or health care nature and includes products such as, for example, feminine hygiene products (i.e., liners, tampons, pads, etc.), incontinence articles, and the like. Tissues, wipes, wound care products (i.e., bandages, gauze pads, etc.), and other types of sanitary products may also be considered as personal care products. Many types of these products are typically packaged in individual sealed packs or pouches and it is often desirable or necessary to store or carry a limited number of such pouches. For example, it is desirable to be able to conveniently store and dispense a limited number of such products when traveling, at the work place, etc. In the case of feminine hygiene products, it is desirable that a consumer have the ability to discretely store and dispense such products.

Various types of “travel packs” or “day packs” of personal care products have been developed in the industry and are commercially available. However, such packages are typically smaller versions of larger packaging systems. For example, various types of soft-side cases, purses, boxes, cartons, etc., are available and marketed for use as portable “packs” for carrying a limited number of personal care products. The conventional packs, however, have certain drawbacks. For example, the size of the case, purse, etc., restricts the number of products that can be carried therein. Also, as the number of products is depleted, the size of the case or purse remains the same and takes up valuable space, particularly when traveling.

The industry is constantly seeking new and improved ways to store and dispense personal care products in a convenient and discrete manner. The present invention relates to just such an improvement.

### SUMMARY

Objects and advantages of the invention will be set forth in part in the following description, or may be obvious from the description, or may be learned through practice of the invention.

The present invention relates to a unique packaging system for personal care products. The packaging system may be used for packaging any type of personal care product, for example wipes, tissues, wound care products, etc., and has particular usefulness for packaging feminine hygiene products such as pads, liners, tampons, etc. All such uses are within the scope and spirit of the invention.

In one embodiment, the packaging system includes an elongated flexible strand of individual sealed pouches. The pouches are connected together in an end-to-end fashion. Each pouch includes at least one type of personal care product carried therein. The strand is “flexible” in that it can be folded along a fold line between the pouches so that the individual pouches lie on top of each other in a stacked configuration.

A separation line is also defined between the individual pouches. This separation line may comprise, for example, a perforation line, score line, etc. The separation line is configured so that a consumer can readily pull and separate a leading pouch from the remaining strand of pouches. It is desirable that the separation line be defined between sealed edges of adjacent pouches. In this manner, the integrity of the pouch is maintained upon separation of the pouch from the remaining strand.

Means are provided for maintaining the pouches in the stacked configuration during, for example, transit and storage of the pouches. The maintaining means permits a consumer to remove and separate a leading pouch from the stack while the remaining portion of the stack is maintained in the stacked configuration. In this manner, the stacked configuration essentially defines the entire size or volume of the packaging system and, thus, as the individual pouches are removed by the consumer, the size of the packaging system decreases accordingly, and the packaging system takes up less and less space and becomes more and more portable and discrete.

In one particularly desirable embodiment, the pouches are soft-sided containers and may be defined by any suitable pliable material, such as a film, film laminate, film/non-woven laminate, etc. In an alternative embodiment, the individual pouches may be formed of a more rigid material, such as cardboard, paper, or plastic, and include a flexible or pliable connection between adjacent pouches so that the pouches can be folded into the stacked configuration.

In one particular embodiment of the packaging system, the strand is folded in an accordion-like fashion.

The means for maintaining the pouches in the stacked configuration may include various devices. In one particular embodiment, a releasable adhesive material is used. The adhesive may be placed between adjacent pouches in the stack, or along the side of the stack. The adhesive material has adhesive and release characteristics to ensure that the pouches remain in the stacked configuration during transit or storage, yet allow a consumer to pull a leading pouch from the stack without tearing or damaging the pouches. Any number of suitable and commercially available adhesives or tacky substances may be used in this regard. In the embodiment wherein the strand of pouches is folded in an accordion-like manner, the adhesive may be placed on opposite faces of adjacent pouches prior to folding the strand into the stacked configuration. In this manner, it is ensured that the adhesive material is placed between each layer of adjacent pouches in the stack.

In an alternative embodiment, the maintaining means may include an adjustable and connectable band member that is disposed and has a length so as to wrap around or encircle the stack. The band member is releasable so that a consumer can disconnect the band in order to remove the leading pouch from the stack. Any configuration of a band or ribbon may be used in this regard. In one embodiment, the band member may include hook and loop fastening material so that the band member will attach to itself regardless of the diminishing size of the stack. In an alternative embodiment, the band member may simply tie together in order to hold the pouches in the stacked configuration. An adhesive may be utilized to releasably hold the band member together around the stack.

In one particular embodiment, the band member is formed integral with or directly connected to the strand of pouches. For example, in one embodiment, the band member may be defined as an extension of one end of the bottom most pouch in the stack. This extension has a length so as to completely wrap around or encircle a full stack and attach to itself or at least to the bottom of the bottom most pouch. As the pouches are depleted, the band member extends further around the stack and attaches to itself. In an alternative embodiment, the band member may be defined by extensions from each end of the bottom most pouch. These extensions have a length so as to at least meet and connect together adjacent to the top most pouch of a full stack. For example, the two ends may simply be ribbons or the like that extend from the bottom most pouch and tie together at the top most pouch. It should be appreciated that a vast number of band or ribbon configurations and attaching devices can be utilized to maintain the pouches in the stacked configuration. All such uses and configurations are within the scope and spirit of the invention.

In accordance with the invention, the packaging system may be particularly configured for packaging and dispensing of feminine hygiene products. This system is essentially as described herein and the pouches would contain at least one type of feminine hygiene product, such as a tampon, pad, liner, and the like.

The invention will be described in greater detail below by reference to embodiments thereof illustrated in the figures.

### BRIEF DESCRIPTION OF THE DRAWINGS

FIG. 1 is a perspective view of a strand of pouched personal care products folded in a stacked configuration according to the invention;

FIG. 2 illustrates the strand of pouched personal care products in a separated state and particularly shows a releasable adhesive between opposite faces of the pouches;

FIG. 3 is a perspective view of an alternative embodiment of the packaging system according to the invention particularly illustrating a wrapping member used to maintain the strand of pouches in a stacked configuration;

FIG. 4 is a perspective view of the embodiment illustrated in FIG. 3 with the wrap member being disconnected in order to remove a pouch from the strand of pouches;

FIG. 5 is a perspective view similar to that of FIG. 4 and particularly illustrates an alternative attaching device for securing the wrap member around the strand of pouches; and

FIG. 6 is an alternative view of the dispensing system particularly illustrating a ribbon or tieable wrap member used to maintain the stack of pouches in a stacked configuration.

### DETAILED DESCRIPTION

Reference will now be made in detail to one or more embodiments of the invention as illustrated in the drawings. Each embodiment is provided by way of explanation of the invention, and is not meant as a limitation of the invention. For example, features illustrated or described as part of one embodiment may be used with another embodiment to yield still a further embodiment. It is intended that the present invention include these and other modifications and variations as come within the scope and spirit of the invention.

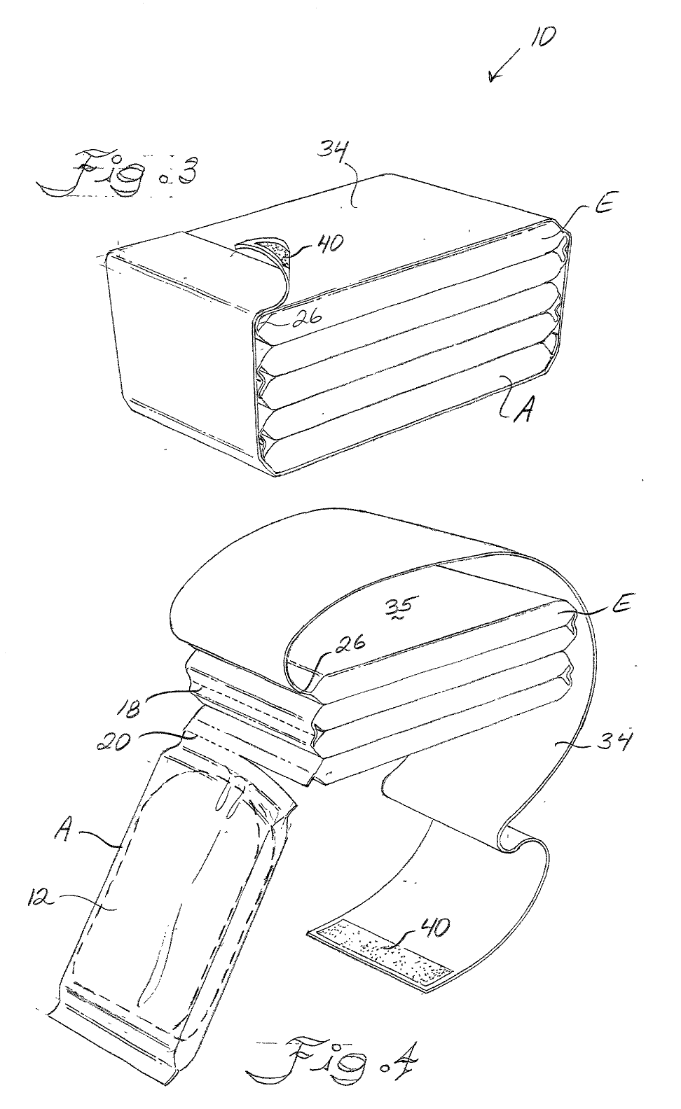

A unique packaging system 10 for personal care products is illustrated in the Figures. As mentioned, “personal care products” may include any manner of personal hygiene or health related products including, for example, feminine hygiene products such as liners, tampons, pads, etc., tissues, wipes, wound care products such as bandages, gauze pads, etc., and the like. The present packaging system has application for storing and dispensing all such types of products and is particularly useful and beneficial for the packaging of feminine hygiene products. In the illustrated embodiment, such a feminine hygiene product 12 (FIG. 4) is graphically illustrated as contained within an individual pouch.

In the illustrated embodiment, the individual pouches A-E are connected in an end-to-end fashion to form a strand 14 of such pouches. The pouches A-E are foldable along a fold line 18 defined between adjacent pouches. The individual pouches are also separable along a separation line 20 that may coincide with the fold line 18. For example, the separation line 20 may be defined by a scored or perforated line that also defines the fold line between adjacent pouches. In this manner, a consumer can readily separate a leading pouch from the remaining strand of pouches 14 simply by pulling or tearing the lead pouch along the separation line 20.

The pouches A-E may be formed from any conventional material. For example, in one particularly desirable embodiment, the pouches are soft sided and may be defined by any suitable pliable material, such as a film, film laminate, film/non-woven laminate, etc. Such materials are well known and widely used in the art. In an alternative embodiment, the pouches may be defined by a more rigid type of material, such as a stiff paper, cardboard, plastic, etc. With this embodiment, the pouches are still “flexible” along the fold line 18 between adjacent pouches so that the pouches can still be folded into a stacked configuration.

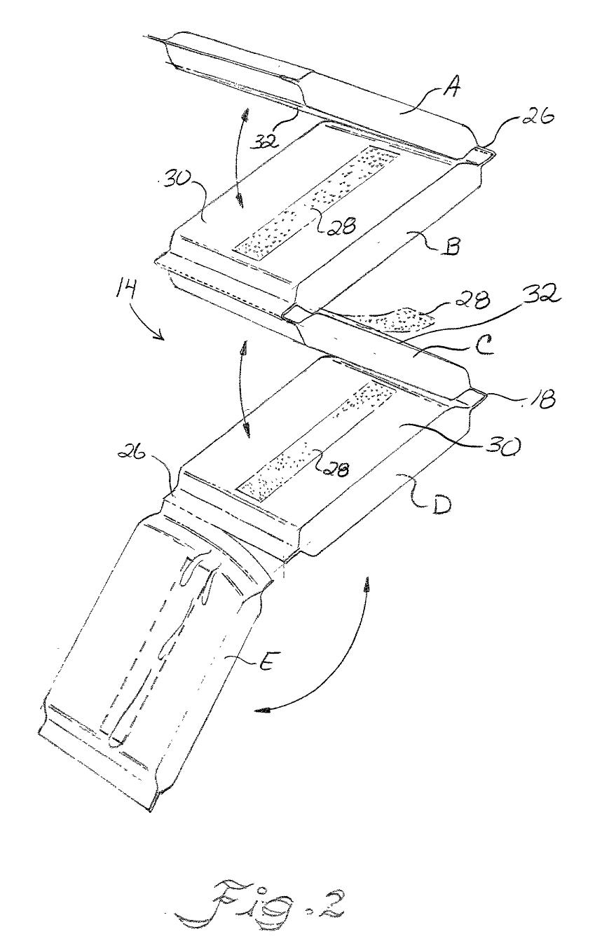

As shown in the illustrated embodiments, it may be desired that the perforation line be defined between adjacent sealed end flaps 26 of the respective pouches. In this manner, as illustrated in FIGS. 2 and 4, a leading pouch can be separated along the separation line 20 from the remaining strand of pouches while maintaining the integrity of the pouches. In other words, it is not necessary to open the pouch being separated or any one of the remaining pouches in order to remove or dispense one of the pouches from the strand 14 of pouches.

As shown in the Figures, the elongated strand of pouches 14 are folded such that the individual pouches A-E are in a stacked configuration. One way to accomplish this as illustrated in the drawings, is to fold the pouches A-E in an accordion-like manner.

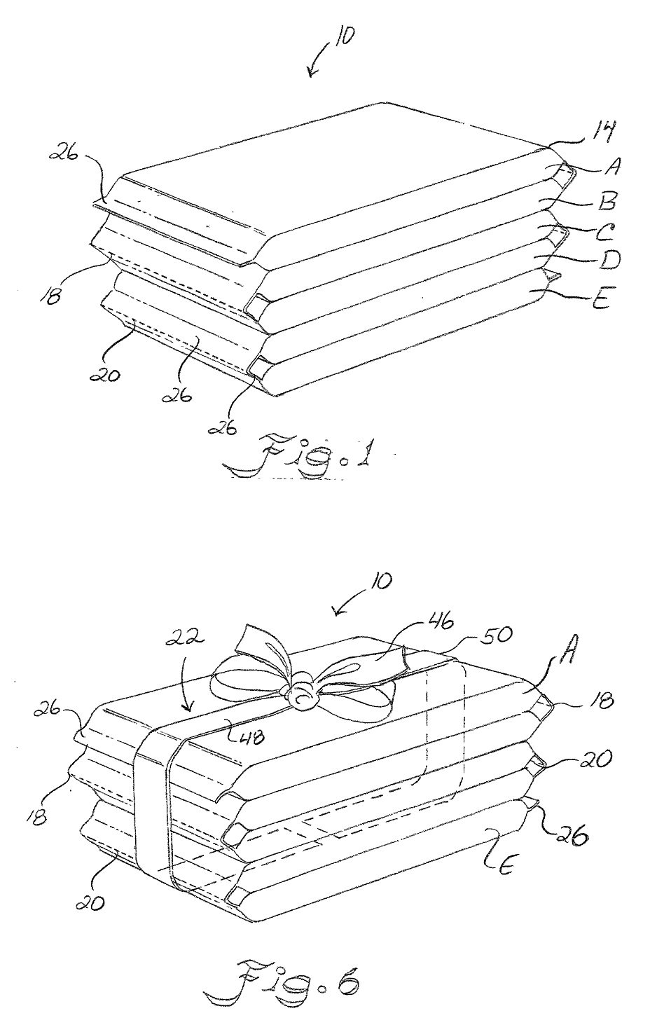

Once folded, maintaining means, generally 22, are provided to hold or secure the pouches in the stacked configuration during transit and storage. The maintaining means 22 permit a consumer to readily remove and separate a leading pouch from the remaining stack of pouches. Various embodiments of the maintaining means are possible. For example, the embodiment of FIGS. 1 and 2 utilizes a releasable adhesive 28 deposited on opposite faces 30 and 32 of adjacent pouches. The adhesive 28 may be applied in generally any pattern on the faces 30, 32. By applying the adhesive 28 on opposite faces 30, 32, it is ensured that at least some adhesive 28 is disposed between every occurrence of opposite faces of adjacent pouches, as can be particularly seen from FIG. 2. Any suitable releasable adhesive can be used in this regard. The adhesive material should have adhesive and release characteristics to ensure that the pouches remain in the stacked configuration of FIG. 1 during transit or storage of the products, yet allow a consumer to pull a leading pouch from the stack without tearing or damaging the pouch material of the pouch being separated or the remaining pouches in the stack. Any number of suitable and commercially available adhesive or tacky substances may be used in this regard.

In the embodiment of FIG. 1 wherein an adhesive 28 is applied between the pouches A-E, a consumer can remove a “leading” pouch from either end of the stack. In other words, pouch A may be the leading pouch, or pouch E may be the leading pouch.

In an alternate embodiment, the adhesive material may be applied along any side of a stack of the pouches after the stack has been formed. This embodiment may be desired from a manufacturing standpoint in that it would not be necessary to apply the adhesive between the pouches prior to folding the pouches into the stacked configuration.

In an alternative embodiment illustrated in FIGS. 3 and 4, a wrapping member or band 34 may be used to maintain the pouches A-E in a stacked configuration. It should be appreciated that countless variations of wrapping members such as bands, ribbons, belts, and respective attaching devices may be utilized in this regard. In the illustrated embodiment, wrapping band 34 is a single band that extends from the end flap 26 of a bottom most pouch E, as particularly seen in FIG. 4. The band 34 has a length so as to completely encircle a full stack of pouches and attach either to itself, as in the illustrated embodiment, or attach to the outer face 35 of pouch E. Referring to FIG. 4, it can be seen that the band 34 is wrapped clockwise around the stack of pouches and thus attaches to itself as illustrated in FIG. 3. It should be understood that the band 34 could also wrap counterclockwise around the stack and have a length so as to attach directly to face 35 of pouch E.

A device is provided with the band member 34 to hold the band in place and thus maintain the integrity of the stack of pouches A-E. Any number of devices or mechanisms may be used in this regard. For example, in the illustrated embodiment, a releasable adhesive 40 is used and disposed as a strip across the leading end of the band 34. This embodiment may be desirable in that the band 34 can easily accommodate the diminishing size of the stack as individual pouches are removed by the consumer. As the stack diminishes, the band 34 is simply drawn further around the stack and the adhesive 40 easily attaches to the band at any position.

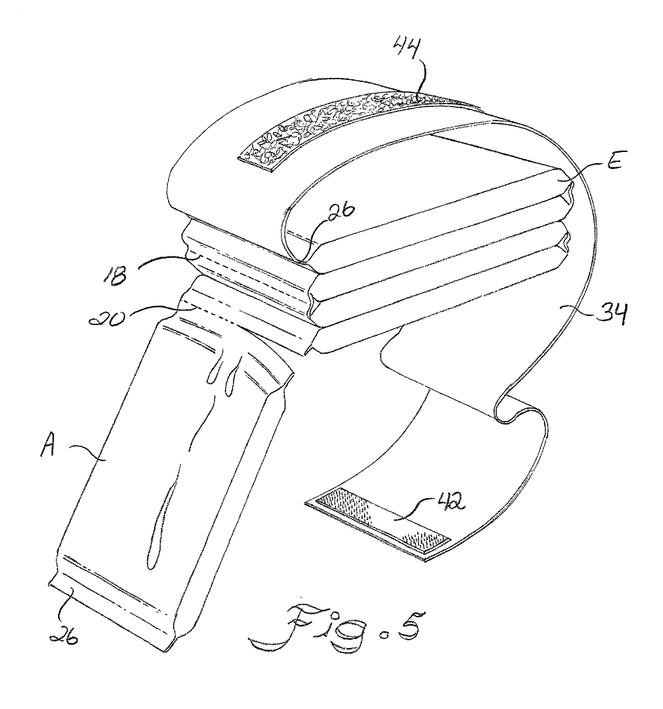

FIG. 5 illustrates an alternative embodiment for attaching the band 34. In this embodiment, a conventional hook and loop fastening material is utilized. For example, a strip of hook material 42 may be disposed across the leading edge of the band 34 and a longitudinally extending strip 44 of loop material may be provided on the opposite face of the band 34. Thus, as the size of the stack diminishes, the band can be drawn further around the stack and attached anywhere along the loop material 44. In an alternate embodiment, the band material itself may be formed of a “loop” material or other type of material to which the hook strip of material 42 readily attaches.

An alternative embodiment of a wrapping band or ribbon is illustrated in FIG. 6. In this embodiment, a relatively simple tieable ribbon 46 is provided. The ribbon has a first half 48 and a second half 50. The halves may be a single continuous element that is attached to a bottom face of pouch E so that the ribbon is not lost upon untying the ribbon to remove the leading pouch A. Thus, in this embodiment, the wrapping band or ribbon may be considered as extending from both ends of the bottom most pouch E for securing adjacent to the top pouch A. The halves 48 and 46 have a sufficient length so that they may be connected together adjacent to pouch A for a full stack of pouches. Any manner of connecting device may be used to releaseably connect the halves 46 and 48. For example, the halves may be simply tied as illustrated in FIG. 6. In an alternative embodiment, an adhesive, hook and loop fastener, mechanical fastener, etc. may be used to releasably connect the halves 46 and 48 together.

It should be appreciated that modifications and variations can be made to the embodiments of the unique packaging and dispensing system described herein without departing from the scope and spirit of the invention. The present invention includes such modifications and variations as come within the scope of the appended claims and their equivalents.

What is claimed is:

1. A packaging system for personal care products, said system comprising:
an elongated flexible strand of individual sealed pouches connected together in an end-to-end fashion, said pouches carrying at least one type of personal care product therein;
said strand folded such that individual said pouches are in a stacked configuration;
a separation line defined between individual said pouches wherein a consumer can readily separate a leading said pouch from the remaining said strand;
means for maintaining the pouches in said stacked configuration during transit and storage while permitting a consumer to remove and separate a leading pouch from said stack; and
wherein said stacked configuration thus defines the size of said packaging system and reduces in size as individual pouches are removed from said stack.

2. The packaging system as in claim 1, wherein said pouches contain a feminine hygiene personal care product.

3. The packaging system as in claim 2, wherein said personal care product is at least one of a tampon, panty liner, or pad.

4. The packaging system as in claim 1, wherein said pouches are soft-sided and defined by a pliable material.

5. The packaging system as in claim 1, wherein said separation line comprises one of a perforation and score line defined between end flaps of said pouches such that separation along said separation line does not breach the integrity of the individual sealed pouches.

6. The packaging system as in claim 1, wherein said strand is folded in an accordion-like manner.

7. The packaging system as in claim 1, wherein said maintaining means comprises a releasable adhesive material disposed so as to maintain adjacent pouches in said stack, said adhesive material allowing separation of said pouches upon a consumer pulling a leading said pouch from said stack.

8. The packaging system as in claim 7, wherein said adhesive is disposed between said pouches.

9. The packaging system as in claim 8, wherein said strand is folded in an accordion-like manner, said adhesive disposed on opposite faces of adjacent said pouches.

10. The packaging system as in claim 7, wherein said adhesive is disposed along a side of said stack.

11. The packaging system as in claim 1, wherein said maintaining means comprises a wrapping band member disposed to encircle said stack and releasably attach to itself.

12. The packaging system as in claim 11, wherein said band member is defined as an extension of one end of a bottommost pouch in said stack.

13. The packaging system as in claim 11, wherein said band member is defined as extensions from both sides of a bottommost pouch in said stack, each said extension having a length so that said extensions jointly encircle said stack and connect together.

14. A packaging system for feminine hygiene products, said system comprising:
an elongated flexible strand of individual sealed pocket pouches connected together in an end-to-end fashion, each said pouch carrying at least one feminine hygiene product therein;
said strand folded such that individual said pouches are in a stacked configuration;
a separation line defined between individual said pouch;
an adhesive material disposed between adjacent said pouches in said stack, said adhesive material maintaining the pouches in said stacked configuration during transit and storage while permitting a consumer to remove and separate a leading pouch from said stack at said pouch's respective separation line; and
wherein said stacked configuration thus defines the size of said packaging system and reduces in size as individual pouches are removed from said stack.

15. The packaging system as in claim 14, wherein said strand is folded in an accordion-like manner, said adhesive disposed on opposite faces of adjacent said pouches.

16. A packaging system for feminine hygiene products, said system comprising:
an elongated flexible strand of individual sealed pocket pouches connected together in an end-to-end fashion, each said pouch carrying at least one feminine hygiene product therein;
said strand folded such that individual said pouches are in a stacked configuration;
a separation line defined between individual said pouch;
a releasable wrapping band member formed integral with said strand and configured with a length so as to encircle said stack to maintain said pouches in said stacked configuration during transit and storage while permitting a consumer to release said band member to remove and separate a leading pouch from said stack at said pouch's respective separation line; and
wherein said stacked configuration thus defines the size of said packaging system and reduces in size as individual pouches are removed from said stack.

17. The packaging system as in claim 16, wherein said band member is formed integral with a bottommost pouch in said stack.

18. The packaging system as in claim 17, wherein said band member comprises an extension of one end of said bottommost pouch member.

19. The packaging system as in claim 17, wherein said band member comprises an extension of each end of said bottommost pouch member.

20. The packaging system as in claim 19, wherein said extensions have a length so as to meet and connect adjacent the topmost pouch in said stack.
